(19) United States
(12) Patent Application Publication
(10) Pub. No.: US 2021/0290803 A1
(43) Pub. Date: Sep. 23, 2021

(54) USE OF GAS MIXTURES COMPRISING OXYGEN FOR THE PRODUCTION OF OZONE

(71) Applicant: Anacail Limited, Glasgow (GB)

(72) Inventors: David Barton, Glasgow (GB); Hugh Potts, Glasgow (GB)

(21) Appl. No.: 16/330,568

(22) PCT Filed: Sep. 20, 2017
(86) PCT No.: PCT/GB2017/052793

§ 371 Date: Mar. 5, 2019

(30) Foreign Application Priority Data
Sep. 20, 2016 (GB) 1615996.4

(51) Int. Cl. A61L 2/20; C01B 13/11; A61L 2/26

(52) U.S. Cl. CPC A61L 2/202; C01B 13/11; A61L 2/26; A61L 2202/121; A61L 2202/24; A61L 2202/11; A61L 2202/122; C01B 2201/64

(57) ABSTRACT

A method of preparing ozone comprising: providing a gas mixture comprising: a) vol. % to 50 vol. % oxygen; b) 50 vol. % to 95 vol. % of an oxide gas; and subjecting the gas mixture to an ozone generating process to produce ozone is disclosed as well as the use of a composition comprising a) 5 vol. % to 50 vol. % oxygen; b) 50 vol. % to 95 vol. % of an oxide gas; as reactant in the preparation of ozone.

### FIELD OF THE INVENTION

The present invention relates to a method of producing ozone. More particularly, the present invention relates to method of producing ozone using a gas mixture comprising oxygen and oxide gases and the use of specific gas mixtures as feedstock in an ozone generating process to convert oxygen to ozone.

### BACKGROUND

Ozone has been used in order to reduce the bioburden in foodstuff, medical devices and water sanitization for many years. As a strong oxidizing agent, ozone is capable of inactivating a wide range of microorganisms and has been found difficult for microorganisms to build up resistance to.

A key advantage of ozone is that it is created from oxygen gas and decays naturally back to oxygen leaving no other residues. This renders ozone environmentally benign compared to other compounds or technologies used alternatively. However, this natural decay of ozone prevents remote facility production with transport of ozone to the point of use. Consequently, ozone is typically generated on demand and in situ.

Most applications for ozone require volumetric concentrations of ozone gas of above 10,000 ppm to provide sufficient amounts of ozone to obtain the desired effect. For example, a medical device sterilizer may use an ozone exposure at 30,000 to 40,000 ppm for 4 hours to sufficiently sterilize reusable medical devices. However, there are also applications which allow for lower ozone concentrations. Ozone generators used for water sanitization use pure oxygen and generate similar concentrations in the gas phase before the resulting ozone is introduced into the water.

In order to achieve such high ozone concentrations, the ozone generators typically use high (kV) voltage corona, dielectric barrier (DBD), similar discharges or ionizing radiation which operate in pure oxygen. Generating a diffuse plasma state in oxygen, a proportion of the oxygen molecules are broken down to then recombine to yield the ozone.

The combination of high voltage sources, plasma but also pure oxygen itself present a fire and explosion hazard which has to be controlled by adopting suitable procedures and equipment. This poses a significant disadvantage which can prevent the use of ozone in certain applications or at certain plants where this fire risk is unacceptable.

It has been attempted to alleviate this problem by diluting oxygen with nitrogen or argon or even by using air. However, ozone concentrations resulting from these gas mixtures were reduced significantly due in part to side reactions of singlet oxygen with nitrogen to form NOx.

Another method for reducing the bioburden employs ionizing radiation sterilization, for example ultraviolet, gamma, electron beam or X ray irradiation. However, certain materials especially when frequent sterilisation is required can be damaged. Typically, ozone is created as a by-product in these processes. Increase in the ozone concentration may lead to an improved and accelerated sterilization and could reduce the frequency and intensity of irradiation treatment.

It is, therefore, an object of the present invention to obviate or mitigate at least one or more of the aforementioned problems.

It is a further object of the present invention to provide a safer method of producing ozone from oxygen comprising gas mixtures in which flammable material is less likely to combust than in pure oxygen while the ozone yields are comparable.

It is a further object of the present invention to provide an ionizing radiation method for sterilization that minimizes or even avoids damage to the materials that are being sterilized.

### SUMMARY OF THE INVENTION

According to a first aspect of the present invention there is provided a method of preparing ozone comprising:
providing a gas mixture comprising
   a) 5 vol. % to 50 vol. % oxygen;
   b) 50 vol. % to 95 vol. % of an oxide gas; and
subjecting the gas mixture to an ozone generating process to produce ozone.

The present invention may therefore be used for treating materials and devices for modification of surface properties (surface energy, oxygen incorporation to improve adhesion for example) or to inactivate microorganisms on a medical or life science device.

The increasing sophistication of prior art devices incorporating advanced polymers, electronic component, optical components, batteries etc. renders such devices highly flammable when placed in an oxygen enriched atmosphere.

The present invention reduces or removes these flammability issues by using a gas mixture which contains up to, for example, 5 vol. % to 50 vol. % oxygen with the remaining component being made up of an oxide containing gas such as carbon dioxide, nitrogen dioxide, and others referred to in the application.

These gas compositions have ozone yields which are considerably less than those achievable with other mixtures which contain highly enriched oxygen. Nevertheless, the ozone concentrations achieved are sufficient for the applications proposed herein.

For the oxygen contents claimed in the present invention there is the intention of reducing flammability.

The gas mixture may comprise less than 30 vol. %, less than 20 vol. %, or 5 vol. % to 30 vol. %, or 5 vol. % to 20 vol. %.

The oxide gas may be selected from the group consisting of carbon monoxide, carbon dioxide, nitrous oxide, nitric oxide, nitrogen dioxide and any mixtures thereof.

The oxide gas may be selected from carbon dioxide, nitrous oxide or any mixtures thereof.

The ozone generating process may be selected from dielectric barrier discharge, corona discharge, ionizing irradiation or cold plasma.

The gas mixture may comprise:
a) 15 vol. % to 25 vol % of oxygen; and
b) 75 vol. % to 85 vol. % of an oxide gas;
with the proviso that these two percentages add up to 100 vol. %.

The gas mixture may comprise:
a) 15 vol. % to 25 vol. % of oxygen; and
b) 75 vol. % to 85 vol. % of an oxide gas selected from carbon dioxide, nitrous oxide and any mixtures thereof;
with the proviso that these two percentages add up to 100 vol. %.

There may also be the step further comprising the sterilizing of an article with the resulting ozone.

The article may be a medical device.

According to a second aspect of the present invention there is provided use of a composition comprising:
a) 5 vol. % to 50 vol. % oxygen;
b) 50 vol. % to 95 vol. % of an oxide gas;
as reactant in the preparation of ozone.

The composition may be a feedstock.

The feedstock may be located in an automated endoscope reprocessor.

The oxide gas may be selected from the group consisting of carbon monoxide, carbon dioxide, nitrous oxide, nitric oxide, nitrogen dioxide and any mixtures thereof.

The oxide gas may be selected from carbon dioxide, nitrous oxide or any mixtures thereof.

The ozone may be prepared using an ozone generating process selected from dielectric barrier discharge, corona discharge, ionizing irradiation or cold plasma.

The composition may comprise:
a) 15 vol. % to 25 vol. % of oxygen; and
b) 75 vol. % to 85 vol. % of an oxide gas selected from carbon dioxide, nitrous oxide or any mixtures thereof;
with the proviso that these two percentages add up to 100 vol. %.

### BRIEF DESCRIPTION OF THE FIGURES

Embodiments of the present invention will now be described, by way of example only, with reference to the accompanying drawings in which:

FIG. 1 is a representation of the ozone concentration obtained from the different mixtures is depicted against the increasing amount of carbon dioxide or nitrogen in the gas mixture (reactant).

### DETAILED DESCRIPTION

Surprisingly it has been found that a method of preparing ozone comprising: providing a gas mixture comprising 5 vol. % to 50 vol. % oxygen, 50 vol. % to 95. vol % of an oxide gas, and subjecting the gas mixture to an ozone generating process to produce ozone, alleviates the aforementioned problems.

### Ozone Generating Processes

Methods useful for the production of ozone according to the present invention using the specific oxygen containing gas mixtures are among others dielectric barrier discharge, corona discharge; ionizing irradiation, for example in the form of ultraviolet light, gamma, electron beam or X ray irradiation; and cold plasma. The corona discharge method uses a power supply to produce an electrical discharge across a dielectric, and an air gap. The dielectric is used to diffuse the discharge across a large area. The oxygen molecules passing through the air gap are exposed to the electrical discharge and are split into ozone. For production of ozone using ultraviolet light, an oxygen containing carrier gas is passed over an ultraviolet light emitting lamp. In the cold plasma method, oxygen containing gas is exposed to a plasma created by dielectric barrier discharge. The oxygen molecules are split into single atoms which then recombine in triplets to form ozone. Ozone may further be formed from oxygen by electrical discharges and by action of high energy electromagnetic radiation.

In a preferred embodiment, the ozone generating process is dielectric barrier discharge.

In embodiments, the gas mixture used for the production of ozone in accordance with the present invention comprises oxygen in an amount of 5 to 50 vol %. The reduced oxygen content leads to a significant reduction in the flammability of the gas mixture and therefore allows for a wider use. In a preferred embodiment, the gas mixture comprises 10 to 30 vol. % of oxygen and in a most preferred embodiment 15 to 25 vol. %.

As a further component, the gas mixture used in the present invention comprises oxide gas. The oxide gas is preferably selected from the group consisting of carbon monoxide (CO), carbon dioxide (CO2), nitrous oxide (N₂O), nitric oxide (NO), nitrogen dioxide (NO₂) and any mixtures thereof. In a preferred embodiment, the gas mixture comprises carbon dioxide, nitrous oxide or any mixture thereof as the oxide gas.

These oxide gases contribute to the formation of ozone in the ozone generating process, as these gases readily give up oxygen atoms thus contributing to the ozone yield.

In embodiments, the gas mixture comprises 50 vol. % to 95 vol. % of oxide gas, in a preferred embodiment 70 vol. % to 90 vol. % and in a most preferred embodiment 75 vol. % to 85 vol. %.

In a preferred embodiment, the gas mixture consists of oxygen and oxide gas.

In an embodiment, the gas mixture comprises:
a) 5 vol. % to 50 vol. % oxygen; and
b) 50 vol. % to 95 vol. % of an oxide gas;
with the proviso that these two percentages add up to 100 vol. %.

In a further embodiment, the gas mixture comprises:
a) 10 vol. % to 30 vol. % oxygen;
b) 70 vol. % to 90 vol. % of an oxide gas selected from the group consisting of carbon monoxide, carbon dioxide, nitrous oxide, nitric oxide, nitrogen dioxide and any mixtures thereof;
with the proviso that these two percentages add up to 100 vol. %.

In a further embodiment, the gas mixture comprises:
a) 15 vol. % to 25 vol. % oxygen;
b) 75 vol. % to 85 vol. % of an oxide gas;
with the proviso that these two percentages add up to 100 vol. %.

In a further embodiment, the gas mixture comprises:
a) 15 vol. % to 20 vol. % oxygen;
b) 75 vol. % to 85 vol. % of an oxide gas selected from carbon dioxide, nitrous oxide or any mixtures thereof;
with the proviso that these two percentages add up to 100 vol. %.

In a further embodiment, the method of preparing ozone further comprises the step of sterilizing an article with the resulting ozone. The article may be a medical device such as endoscopes, sets of instruments, implantable devices, life science consumables and single use devices such as microtitre plates, syringes, dressings, disposable blades, disposable scissors, disposable needles; sample collection tubes, preparation vessels such as centrifuge cubes; cell culture equipment such as flasks, dishes and bioreactors; biological equipment such as all appliances for peptide and protein preparation including powdered media.

Further, the article may be a contaminated space such as fume cupboards, glove boxes and other equipment where contamination can be an issue. Contamination in the sense of the present invention relates to biological contamination by microorganisms such as bacteria, viruses, yeasts, moulds, spores, vegetative cells and parasites.

Further, the article may be a foodstuff in food processing to reduce the bioburden and to increase shelf life. For example, fruit and vegetables may be treated with ozone in food purifiers or washers to improve food surface hygiene. But also sanitation of food plant equipment, reuse of waste water, lowering of biological oxygen demand and chemical oxygen demand of food plant waste can be achieved with ozone generated according to the present invention. In this regard, waste water treatment is not restricted to such of the food industry.

Further, ozone generated according to the present invention can be used for disinfecting meat based foodstuffs and production plants and wastes such as recycled poultry chill water and disinfection of poultry carcasses.

Further, the method of the present invention may be used for decontaminating semi enclosed drain systems. A cap may be placed over for example a semi enclosed drain with the cap comprising an ozone generator. The gas mixture or composition is pumped into the trapped volume between the cap and the water sump of the drains and the ozone is generated within this trapped volume between the cap and the water sump.

Sterilization may be carried out by placing the article directly into the reaction chamber where ozone is generated from the reactants/gas mixture or providing the article in a sealed pack which comprises the gas mixture before placing the sealed pack into the reaction chamber. This has the added benefit that no further packaging has to be done under sterile conditions.

A further aspect of the present invention is the use of a composition comprising 5 vol. % to 50 vol. % oxygen, 50 vol. % to 95 vol. % of an oxide gas as reactant in the preparation of ozone.

Reactant in the sense of the present invention is to be understood as the chemical material that undergoes chemical reaction to yield the ozone.

The composition can be provided to the reaction chamber as a feedstock, i.e. a premix of the different components which will form the gas mixture within the reaction chamber for preparation of the ozone. The composition may be provided as a premix in a cylinder that can easily be transported and stored. The composition may be stored and/or transported as a liquefied composition or in the gaseous state. Further, the different components of the composition can be added through different channels to be mixed only within the reaction chamber before being subjected to the ozone generating process.

The feedstock may further be provided directly within a medical device. For example, the feedstock may be located in an automated endoscope reprocessor. The ozone required for sterilisation of the endoscope can be produced directly within the reprocessor.

The composition may be the gas mixture as described with regard to the present invention. The feedstock can either be in the gaseous aggregation state or liquefied.

As ozone generating process any of the processes mentioned in the present application can be used.

In a preferred embodiment, the composition used as reactant in the preparation of ozone comprises 15 vol. % to 25 vol. % of oxygen and 75 vol. % to 85 vol. % of an oxide gas selected from carbon dioxide, nitrous oxide or any mixtures thereof, with the proviso that these two percentages add up to 100 vol. %.

### EXAMPLES

### Example 1

Using a 60 mm diameter electrode, a dielectric barrier discharge was generated at 100% duty cycle, 21 kHz supply frequency and 3.80 kV electrode voltage. A range of gas mixtures were prepared containing different ratios of oxygen and carbon dioxide gases. The gas mixtures were prepared in sealed polythene bags which were then subjected to the dielectric barrier discharge to generate the ozone within the bag.

A discharge was run for a fixed 60 s period, after which the ozone concentration was measured using a 2B Technologies 106-MH ozone meter.

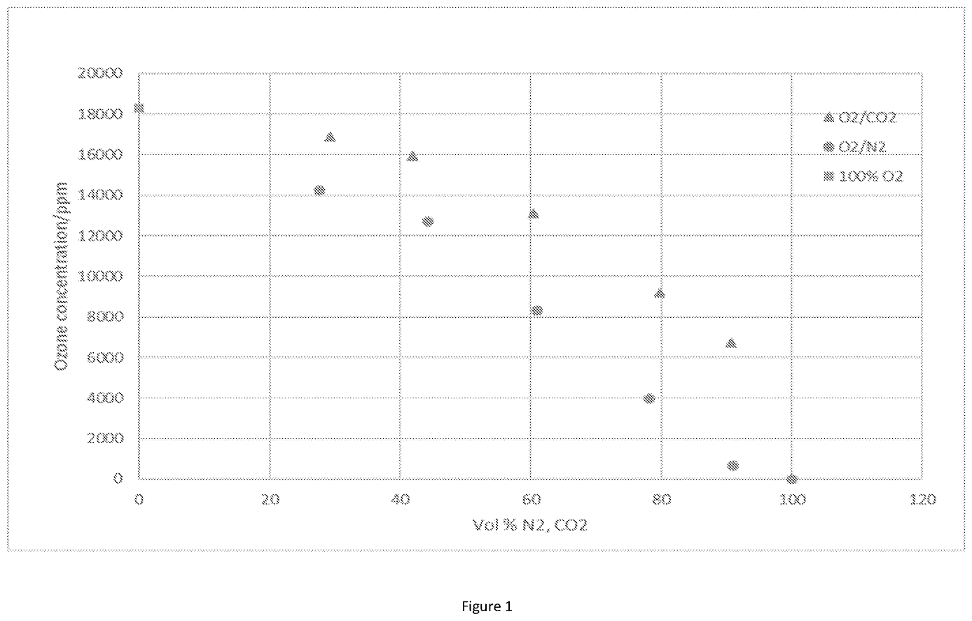

In FIG. 1, the ozone concentration obtained from the different mixtures is depicted against the increasing amount of carbon dioxide or nitrogen in the gas mixture (reactant).

As shown in FIG. 1 and as expected, the ozone concentration decreases with lower amounts of oxygen in the reaction chamber. However, as the oxide gases contribute to the ozone yield, even with lower oxygen concentrations sufficient amounts of ozone can be produced at lower oxygen content in the gas mixture as compared to gas mixtures comprising oxygen and nitrogen.

Whilst specific embodiments of the present invention have been described above, it will be appreciated that departures from the described embodiments may still fall within the scope of the present invention. For example, any suitable amount of oxygen may be used.

1. A method of preparing ozone comprising:
providing a gas mixture comprising:
a) 15 vol. % to 25 vol. % oxygen;
b) 75 vol. % to 85 vol. % of an oxide gas,
wherein the oxide gas is selected from the group consisting of carbon dioxide, nitrous oxide, nitrogen dioxide and any mixtures thereof,
with the proviso that the two percentages for the gas mixture add up to 100 vol. %;
subjecting the gas mixture to an ozone generating process to produce ozone; and
sterilizing a medical or life science device with the ozone, wherein the sterilizing comprises placing the medical or life science device directly into an ozone containing reaction chamber the ozone being generated from the gas mixture or providing the medical or life science device in a sealed pack that comprises the gas mixture before placing the sealed pack into the reaction chamber.

2.-3. (canceled)

4. The method of preparing ozone according to claim 1, wherein the oxide gas is selected from carbon dioxide, nitrous oxide or any mixtures thereof.

5. The method of preparing ozone according to claim 1, wherein the ozone generating process is selected from dielectric barrier discharge, corona discharge, ionizing irradiation or cold plasma.

6.-9. (canceled)

10. A method for preparing ozone used to sterilize a medical or life science device, the method comprising:
sterilizing a medical or life science device with ozone, wherein a gas mixture is a reactant in the preparation of ozone and the gas mixture comprises:
a) 15 vol. % to 25 vol. % oxygen;
b) 75 vol. % to 85 vol. % of an oxide gas;
with the proviso that the two percentages for the gas mixture add up to 100 vol. %,
wherein the sterilizing comprises placing the medical or life science device directly into an ozone containing reaction chamber the ozone being generated from the gas mixture or providing the medical or life science device in a sealed pack that comprises the gas mixture before placing the sealed pack into the reaction chamber, and
wherein the oxide gas is selected from the group consisting of carbon dioxide, nitrous oxide, nitrogen dioxide and any mixtures thereof.

11. The method according to claim 10, wherein the gas mixture is a feedstock that is a premix of different components that will form the gas mixture within the reaction chamber for the preparation of the ozone.

12. The method according to claim 10, wherein the gas mixture is a feedstock that is located in an automated endoscope reprocessor.

13. (canceled)

14. The method according to claim 10, wherein the oxide gas is selected from carbon dioxide, nitrous oxide or any mixtures thereof.

15. The method according to claim 10, wherein ozone is prepared using an ozone generating process selected from dielectric barrier discharge, corona discharge, ionizing irradiation or cold plasma.

16. (canceled)

17. The method according to claim 10, wherein the medical or life science device is selected from endoscopes, sets of instruments, implantable devices, life science consumables and single use devices such as microtitre plates, syringes, dressings, disposable blades, disposable scissors, disposable needles; sample collection tubes, preparation vessels such as centrifuge cubes; cell culture equipment such as flasks, dishes and bioreactors; biological equipment such as all appliances for peptide and protein preparation including powdered media.

18. The method according to claim 10, wherein the medical or life science device is contaminated space including fume cupboard and glove boxes.

19. The method according to claim 10, wherein the ozone is used for decontaminating semi enclosed drain systems.

20. The method according to claim 10, wherein the sterilizing comprises placing the medical or life science device directly into the reaction chamber where ozone is generated from the gas mixture.

21. The method according to claim 10, wherein the sterilizing comprises providing the medical or life science device in a sealed pack which comprises the gas mixture before placing the sealed pack into the reaction chamber.
